(19) United States
(12) Patent Application Publication
(10) Pub. No.: US 2010/0029974 A1
(43) Pub. Date: Feb. 4, 2010

(54) Catalytic system and method for oxidative carbonylation reaction

(75) Inventors: Chih-Wei Chang, Taipei City (TW); Chia-Jung Tsai, Taipei City (TW); Ying-Tien Chen, Taipei City (TW)

Correspondence Address: EDWARDS ANGELL PALMER & DODGE LLP, P.O. BOX 55874, BOSTON, MA, 02205, US

(73) Assignee: China Petrochemical Development Corporation, Taipei City (TW)

(21) Appl. No.: 12/315,789

(22) Filed: Dec. 4, 2008

(30) Foreign Application Priority Data
Jul. 31, 2008 (TW) 097128943

(51) Int. Cl. C07C 69/96; B01J 31/02

(52) U.S. Cl. 558/277; 502/167; 502/165

(57) ABSTRACT

A catalytic system for an oxidative carbonylation reaction is provided, which includes a metal organohalogen catalyst, at least one organic nitrogen-containing heterocyclic adjuvant, and an inorganic co-catalyst, wherein the inorganic co-catalyst is selected from carboxylates, nitrates, halides, oxides, and complexes of lead, lanthanum, titanium, tungsten, and dysprosium. The process for producing a dialkyl carbonate by performing a liquid-phase oxidative carbonylation reaction of an alcohol in the presence of the catalytic system is significantly improved, and the conversion and selectivity of the catalytic reaction are increased.

### FIELD OF THE INVENTION

The present invention relates to catalytic systems for oxidative carbonylation reactions, and more particularly to a catalytic system for oxidatively carbonylating an alcohol in a liquid phase as well as a method for producing dimethyl carbonate by using the same.

### BACKGROUND OF THE INVENTION

It is known that dimethyl carbonate can be used as an organic solvent, or used as a reactant instead of phosgene in the synthesis of other alkyl and aryl carbonates. These alkyl and aryl carbonates are useful substances, and not only utilized as synthetic lubricants, solvents, plasticizers, monomers for organic glasses, etc., but also applied to the processes referring to methylation and carbonylation reactions, for example, in the preparation of isocyanates, polyurethanes, polycarbonates, and so on. Additionally, other applications of dimethyl carbonate are investigated. For example, U.S. Pat. No. 2,331,386 discloses the use of dimethyl carbonate or other organic carbonates, or a mixture of organic carbonate and ether (especially methyl t-butyl ether) as an anti-explosive additive for gasoline or fuels heavier than gasoline. Conventionally, dimethyl carbonate is synthesized by phosgenation of methanol (phosgene route). These processes suffer from numerous problems due to the high toxicity of phosgene and the corrosion of apparatuses, the phosgene route has been gradually replaced by oxidative carbonylation of methanol (oxidative carbonylation route) in recent years. In comparison with the phosgene route, the oxidative carbonylation route has advantages of easy acquirement of starting materials, simple synthetic procedures, less environmental pollution and lower production cost.

The oxidative carbonylation of methanol may basically be classified into two manners: gas-phase synthesis and liquid-phase synthesis. The method of gas-phase synthesis is represented by Ube Industries. Ltd., Japan. For example, U.S. Pat. No. 5,162,563 discloses a process for preparing dimethyl carbonate by using a palladium chloride catalyst with a copper compound thereon to increase the activity of the catalyst. In this reaction system, the concentration of nitrogen monoxide has a significant influence on the yield. The methods developed by Enichem Synthesis S.p.A., Italy are representatives of the liquid-phase synthesis. For example, Euro Patent 0460735 discloses producing dimethyl carbonate via oxidative carbonylation of methanol in the presence of cuprous chloride as a catalyst in an autoclave. U.S. Pat. No. 4,218,391 and U.S. Pat. No. 4,318,862 disclose using metal salts of the group IB, IIB, VIIIB of the periodic table as a catalyst, especially monovalent copper salts such as cuprous bromide, cuprous chloride, and cuprous perchlorate, to synthesize dimethyl carbonate. In order to reach a sufficient reaction rate in the Enichem processes, a high concentration of cuprous chloride must be employed, and it leads to corrosion of facilities. To obviate this defect, reactors lined with anticorrosion materials such as glass are considered, but the use of liners causes difficulties on an industrial scale.

Moreover, methods for making dimethyl carbonate in the presence of metal complex catalysts are studied. For example, U.S. Pat. No. 4,113,762 discloses preparing dimethyl carbonate in the presence of a copper-containing complex catalyst which is formed by the reaction of cuprous chloride with vanadium trichloride, chromium trichloride, iron trichloride, cobalt dichloride, aluminum trichloride, or silicon tetrachloride. U.S. Pat. No. 5,258,541 and U.S. Pat. No. 6,458,914 disclose using a cupric salt together with an alkaline earth metal halide to manufacture alkyl carbonates in order to increase the catalytic activity of a copper halide catalyst. However, the yield of dialkyl carbonate can't be effectively enhanced by the aforementioned methods, and the catalysts used may cause clogging of the reaction equipments.

Therefore, it has been desired to develop a method increasing the conversion of starting materials, the selectivity of catalytic reactions and the yield of products.

To overcome the above-mentioned problems, the present invention has been accomplished after the present inventors made extensive researches and improvements.

### SUMMARY OF THE INVENTION

It is an object of this invention to provide a catalytic system for oxidative carbonylation with high raw material conversion.

Another object of this invention is to provide a catalytic system for oxidative carbonylation with high reaction selectivity.

Further an object of this invention is to provide a catalytic system for oxidative carbonylation with an improved overall yield of products.

To achieve the aforementioned and other objects, a catalytic system for an oxidative carbonylation reaction is provided in the present invention, which comprises a metal organohalogen catalyst, at least one organic nitrogen-containing heterocyclic adjuvant, and an inorganic co-catalyst, wherein the inorganic co-catalyst is selected from a group consisting of carboxylates, nitrates, halides, oxides, and complexes of lead, lanthanum, titanium, tungsten, and dysprosium. The present invention also provides a method for producing a dialkyl carbonate, which comprises performing an oxidative carbonylation reaction in a liquid phase by reacting an alcohol with carbon monoxide and oxygen in the presence of a catalytic system which is composed of a metal organohalogen catalyst, at least one organic nitrogen-containing heterocyclic adjuvant, and an inorganic co-catalyst, wherein the inorganic co-catalyst is selected from a group consisting of carboxylates, nitrates, halides, oxides, and complexes of lead, lanthanum, titanium, tungsten, and dysprosium. In the present method, a metal organohalogen catalyst combined with an organic nitrogen-containing heterocyclic adjuvant and an inorganic co-catalyst is employed so as to increasingly improve the conversion and selectivity of a catalytic reaction as well as the total yield of the reaction.

### DETAILED DESCRIPTION OF THE PREFERRED EMBODIMENTS

The character and efficacy of the present invention will be further described in details by referring to the following examples, but the present invention is not limited thereto.

In the present invention, a catalytic system comprising a metal organohalogen catalyst, at least one organic nitrogen-containing heterocyclic adjuvant, and an inorganic co-catalyst is used for an oxidative carbonylation reaction. Examples of the metal used as a catalyst include the elementals of the group IB, IIB, VIIIB of the periodic table, such as copper (I, II), vanadium (III), chromium (III), iron (III), cobalt (II), aluminum (III), and silicon (IV). Among them, monovalent copper (cuprous) and divalent copper (cupric) are preferred. In one embodiment, a metal organohalogen is employed, for example, cupric halide or cuprous halide is used as a catalyst. Examples of the cupric and cuprous halides include, but not limited to, cupric chloride, cuprous chloride, cuprous bromide, and cuprous iodide. The concentration of the metal organohalogen added is usually in a range of from 1 to 50000 ppm, and preferably in a range of from 2000 to 30000 ppm.

The catalytic system of the present invention further comprises at least one organic nitrogen-containing heterocyclic adjuvant and an inorganic co-catalyst in addition to the metal organohalogen. The organic nitrogen-containing heterocyclic adjuvant can be a five-membered heterocyclic ring compound containing two nitrogen atoms, a benzo-fused five-membered heterocyclic ring compound containing two nitrogen atoms, a six-membered heterocyclic ring compound containing two nitrogen atoms, or a fused cyclic compound containing nitrogen atoms. For example, imidazole compounds having a structure represented by the formula (I):

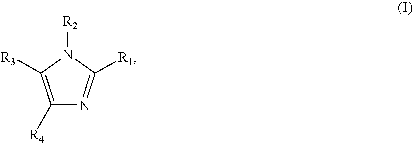

in which R₁, R₂, R₃, and R₄ are independently selected form the group consisting of hydrogen, halogen, nitro, cyano, amino, C₁₋₆alkylamino, C₁₋₁₂alkyl, C₁₋₁₂alkoxy, C₁₋₁₂ alkanoyl, C₃₋₁₂ cycloalkyl, C₃₋₁₂ cycloalkoxy, C₃₋₁₂ cycloalkanoyl, C₆₋₂₀ aryl, C₇₋₂₀ arylalkyl, and C₇₋₂₀ alkylaryl, wherein the C₁₋₆ alkylamino, C₁₋₁₂ alkyl, C₁₋₁₂ alkoxy, C₁₋₁₂ alkanoyl, C₃₋₁₂ cycloalkyl, C₃₋₁₂ cycloalkoxy, C₃₋₁₂ cycloalkanoyl, C₆₋₂₀ aryl, C₇₋₂₀ arylalkyl, and C₇₋₂₀ alkylaryl are optionally substituted with halogen, nitro, and/or cyano. Examples of the imidazole compound include, but not limited to, 2-methylimidazole, 1-methylimidazole, N-acetylimidazole, 2-isopropylimidazole, 1-(4-nitrophenyl)imidazole, and 4,5-diphenylimidazole.

In one embodiment, the catalytic system of the present invention uses an imidazole compound substituted with C₁₋₆ alkyl, C₁₋₆alkanoyl, C₁₋₆alkylamino, and/or phenyl as the organic heterocyclic adjuvant. In the catalytic system of the present invention, the molar ratio of a metal organohalogen catalyst to an organic heterocyclic adjuvant is normally in a range of from 10:1 to 1:10, and preferably in a range of from 5:1 to 1:5.

In the catalytic system of the present invention, the inorganic co-catalyst can be carboxylates, nitrates, halides, oxides, or complexes such as tetra-, penta-, hexa- or octa-coordinated complexes, of lead, lanthanum, titanium, tungsten, or dysprosium. Examples of the inorganic co-catalyst include, but not limited to, tungstic acid, lead nitrate, lanthanum oxide, titanium dioxide, and dysprosium oxide. In general, the amount of an inorganic co-catalyst added is in a range of from 0.001 to 0.5 moles, and preferably in a range of from 0.001 to 0.1 moles.

The method for producing a dialkyl carbonate in the present invention is to carry out an oxidative carbonylation reaction in a liquid phrase by reacting an alcohol having 1 to 6 carbon atoms (e.g. methanol, ethanol, propanol, and butanol) with carbon monoxide and oxygen in the presence of a catalytic system composed of a metal organohalogen, at least one organic nitrogen-containing heterocyclic adjuvant, and an inorganic co-catalyst, wherein the inorganic co-catalyst is selected from a group consisting of carboxylates, nitrates, halides, oxides, and complexes of lead, lanthanum, titanium, tungsten, and dysprosium. Typically, the molar ratio of the metal organohalogen catalyst to the organic heterocyclic adjuvant is in a range of from 10:1 to 1:10, and preferably in a range of from 5:1 to 1:5. Furthermore, the oxidative carbonylation reaction of the present invent is carried out within a temperature between 60 to 200° C., and preferably between 90 to 180° C. As for the reaction pressure in the present invention, the total pressure of the reaction system is usually maintained in a range of from 15 to 40 kg/cm², and preferably in a range of from 20 to 30 kg/cm².

The present invention is illustrated by the following Examples, which must not however be considered as limiting thereof.

### EXAMPLES

Conversion, selectivity and yield referred herein were defined as the following equations:

Conversion (%)=[the amount of methanol converted (mol)/the amount of methanol fed (mol)]×100%

Selectivity (%)=[2×the amount of dimethyl carbonate produced (mol)/the amount of methanol converted (mol)]×100%

Yield (%)=conversion×selectivity×100%

### Comparative Example 1

In a 1 L, Teflon-lined stainless steel high-pressure reactor equipped with an agitator, 228.5 g (7.14 moles) of methanol and 5000 ppm of cuprous chloride (calculated as copper metal) was charged. A nitrogen gas was introduced into the reactor to replace air therein until a pressure of 25 kg/cm² in the reactor was attained. The agitator was actuated and the temperature of the reaction system was heated to 120° C. Then, a gaseous mixture of carbon monoxide having a partial pressure of 23.1 kg/cm² and molecular oxygen having a partial pressure of 1.9 kg/cm² was fed into the reactor to initiate an oxidative carbonylation reaction. In the period of the reaction, the pressure in the reactor was continuously kept at 25 kg/cm². After the reaction was carried out for 80 minutes, products were analyzed by gas chromatography and the conversion, selectivity and yield of the reaction were respectively calculated. The results are shown in Table 1.

### Comparative Example 2

Comparative Example 1 was repeated, but two equimolar portions of N-acetylimidazole acted as an organic adjuvant was additionally added based on the quantity of cuprous chloride. Products were analyzed by gas chromatography and the conversion, selectivity and yield of the reaction were respectively calculated. The results are shown in Table 1.

### Example 1

Comparative Example 2 was repeated, but 0.001 mole of lead nitrate acted as an inorganic co-catalyst was additionally added. Products were analyzed by gas chromatography and the conversion, selectivity and yield of the reaction were respectively calculated. The results are shown in Table 1.

### Examples 2-5

Example 1 was repeated, but various inorganic co-catalysts were applied in accordance with Table 1. Products were analyzed by gas chromatography and the conversion, selectivity and yield of the reaction were respectively calculated. The results are shown in Table 1.

TABLE 1

| | Inorganic | | | |
|---|---|---|---|---|
| Example | co-catalyst | Conversion % | Selectivity % | Yield % |
| Comparative | — | 11.2 | 87.2 | 9.8 |
| Example 1 | | | | |
| Comparative | — | 15.0 | 82.8 | 12.5 |
| Example 2 | | | | |
| Example 1 | Lead nitrate | 16.1 | 92.5 | 14.9 |
| Example 2 | Lanthanum oxide | 18.7 | 90.9 | 17.0 |
| Example 3 | Titanium dioxide | 16.7 | 92.2 | 15.4 |
| Example 4 | Tungstic acid | 16.5 | 85.4 | 14.0 |
| Example 5 | Dysprosium oxide | 16.7 | 81.5 | 13.6 |

As given in Table 1, by using a catalytic system which comprised a metal organohalogen as a catalyst, an organic nitrogen-containing heterocyclic compound as an adjuvant, and an inorganic co-catalyst to carry out an oxidative carbonylation reaction of an alcohol in the liquid phase to manufacture dialkyl carbonate, it can substantially increase the conversion of the alcohol and the selectivity of the reaction as well as the yield of the product.

The features and functions of the present invention have been elucidated in the foregoing detailed descriptions. Those skilled in the art will appreciate that modifications and variations according to the spirit and principle of the present invention may be made. All such modifications and variations are considered to fall within the spirit and scope of the present invention as defined by the appended claims.

What is claimed is:

1. A catalytic system for an oxidative carbonylation reaction, which comprises a metal organohalogen catalyst, at least one organic nitrogen-containing heterocyclic adjuvant, and an inorganic co-catalyst, wherein the inorganic co-catalyst is selected from a group consisting of carboxylates, nitrates, halides, oxides, and complexes of lead, lanthanum, titanium, tungsten, and dysprosium.

2. The catalytic system according to claim 1, wherein the metal organohalogen catalyst is cupric halide and/or cuprous halide.

3. The catalytic system according to claim 1, wherein the metal organohalogen catalyst is selected from the group consisting of cupric chloride, cuprous chloride, cuprous bromide, and cuprous iodide.

4. The catalytic system according to claim 1, wherein the organic heterocyclic adjuvant is selected from the group consisting of a five-membered heterocyclic ring compound containing two nitrogen atoms, a benzo-fused five-membered heterocyclic ring compound containing two nitrogen atoms, a six-membered heterocyclic ring compound containing two nitrogen atoms, and a fused cyclic compound containing nitrogen atoms.

5. The catalytic system according to claim 1, wherein the organic heterocyclic adjuvant is imidazole substituted with a group selected from the group consisting of C₁₋₆ alkyl, C₁₋₆alkanoyl, C₁₋₆alkylamino, and phenyl.

6. The catalytic system according to claim 1, wherein the metal organohalogen catalyst to the organic heterocyclic adjuvant has a molar ratio of from 10:1 to 1:10.

7. The catalytic system according to claim 1, wherein the metal organohalogen catalyst to the organic heterocyclic adjuvant has a molar ratio of from 5:1 to 1:5.

8. The catalytic system according to claim 1, wherein the inorganic co-catalyst is selected from the group consisting of tungstic acid, lead nitrate, lanthanum oxide, titanium dioxide, and dysprosium oxide.

9. The catalytic system according to claim 1, wherein the inorganic co-catalyst is added in an amount of from 0.001 to 0.5 moles.

10. The catalytic system according to claim 1, wherein the inorganic co-catalyst is added in an amount of from 0.001 to 0.1 moles.

11. A method for producing a dialkyl carbonate, which comprises performing an oxidative carbonylation reaction in a liquid phase by reacting an alcohol with carbon monoxide and oxygen in the presence of the catalytic system according to claim 1.

12. The method according to claim 11, wherein the alcohol contains one to six carbon atoms.

13. The method according to claim 11, wherein the alcohol is methanol.

14. The method according to claim 11, wherein the oxidative carbonylation reaction is carried out at a pressure of from 15 to 40 kg/cm².

15. The method according to claim 11, wherein the oxidative carbonylation reaction is carried out at a pressure of from 20 to 30 kg/cm².

16. The method according to claim 11, wherein the oxidative carbonylation reaction is carried out within a temperature range of between 60 and 200° C.

17. The method according to claim 11, wherein the oxidative carbonylation reaction is carried out within a temperature range of between 90 and 180° C.
